(19) United States
(12) Patent Application Publication
(10) Pub. No.: US 2024/0312609 A1
(43) Pub. Date: Sep. 19, 2024

(54) Method and System for Securely Managing Transactions Between Healthcare Stakeholders

(71) Applicant: Old Well Solutions, LLC, San Antonio, TX (US)

(72) Inventors: Eric Rindler, San Antonio, TX (US); Anton Domratchev, San Antonio, TX (US)

(21) Appl. No.: 18/606,679

(22) Filed: Mar. 15, 2024

Related U.S. Application Data
(60) Provisional application No. 63/490,944, filed on Mar. 17, 2023.

(51) Int. Cl. G16H 40/20; G06F 21/31; G16H 10/60

(52) U.S. Cl. CPC G16H 40/20; G06F 21/31; G16H 10/60

(57) ABSTRACT

A computer-implemented method of sharing and restricting data between healthcare stakeholders comprising the steps of requesting an appointment, confirming an appointment, verifying provider information, updating provider information, requesting patient medical history, requesting patient payment information.

### CROSS REFERENCES TO RELATED APPLICATION

Not applicable.

### FEDERALLY SPONSORED RESEARCH OR DEVELOPMENT

Not applicable.

### BACKGROUND OF THE INVENTION

### 1. Field of the Invention

The present invention generally relates to a method for securely sharing patient and non-patient information among healthcare stakeholders using a computer network.

### 2. Description of the Related Art

The provision of healthcare involves more than a healthcare provider simply meeting with a patient to remedy a patient's ailments or prevent ailments in the first place. Healthcare involves several stakeholders: patients, healthcare providers (“providers” such as physicians, dentists, etc.), payors (e.g., insurance carriers), and allied partners (e.g., vendors supporting healthcare services, medical device manufacturers, etc.). Challenges arise with the transfer of information, securitization, maintenance of records, regulatory compliance, etc.

Many healthcare providers and other stakeholders maintain websites, social media accounts, and the like, to promote their services. Many such sites allow patients to schedule appointments and/or inquire about healthcare provider availability.

Many payors maintain databases that patients can review to determine whether a particular healthcare provider is “in network.” The remainder of this Section entitled “Description of the Related Art” identifies examples of problems in the related art.

When a patient requests an appointment from a provider, the provider's website is frequently not current (e.g., provider availability; provider's willingness to accept new patients; provider location; provider hours); the patient is redirected to a new website and/or the patient must call or email the provider's office directly.

At the time of a patient visit, the patient provides a medical history. Each provider has their own form (i.e., not standardized) that the patient fills out, and the provider must have consent (i.e., HIPPA compliance) in place prior to receiving the information. At the first appointment, the provider requires the patient to review and sign one or more agreements (e.g., Business Associate Agreement (“BAA”)), financial policy, etc.

At some point, the provider must verify the patient's insurance. This step usually occurs after treatment is rendered, rarely beforehand. This step often involves call center/fax verification. Patient insurance information is usually delivered at the first appointment and requires subsequent verification.

The insurance provider (more broadly, “payor”) must determine if the provider is still in the payor's network and the payor must determine whether the patient is insured or otherwise a beneficiary of the payor.

The payor must determine and update its records as to whether the provider is accepting new patients; or the provider must update its status with one or more payors.

In the overall healthcare framework, different stakeholders are siloed and almost never able to exchange insurance information and medical history at the time of referring the patient(s) to a provider. And provider information maintained by payors is often out of date and not updated in a timely manner, which leads to a fragmented communication network.

Information is delivered in a series of manual (i.e., fragmented) time-consuming steps, which often leads to unnecessary repetition and clerical errors.

The present invention overcomes these inadequacies and improves the sharing, maintaining, updating, and restricting data between healthcare stakeholders.

### BRIEF SUMMARY OF THE INVENTION

The present invention is a computer-implemented method of sharing and restricting data between healthcare stakeholders comprising the steps of requesting an appointment, confirming an appointment, verifying provider information, updating provider information, requesting patient medical history, requesting patient payment information.

The present invention is superior to other known computer-implemented methods of sharing and restricting data between healthcare stakeholders because: (1) the patient is able to see an up to date list of providers, and the list is provided by the patient's payor or organization working directly with providers; (2) the patient is able to request or book an appointment directly from the list of providers; (3) the patient's medical history is recorded and kept secure by a system of secure data aggregation and distribution (“SOSDAD”) or participating records keeping partners (e.g., MyChart) or a database that keeps information on patient prescriptions (e.g., stated back prescription monitoring programs), and that information is shared with the provider-of-choice with an explicit consent from the patient at the time of appointment booking/acceptance; (4) a BAA/HIPPA agreement is put into place at the time of appointment booking/acceptance between a provider and the patient to facilitate secure patient information transmission; (5) insurance verification happens when a patient consents by using a Single Sign On (SSO) mechanism with their payor portal and consenting to secure insurance information transmission (insurance information passed to provider prior to patient presenting for exam; no need to call or fax/has consent to request info; (6) patient insurance card delivered to provider immediately at the time of appointment booking/acceptance; (7) provider participation is verified by the payor on a regular interval (e.g., 60 days) and provider in-network eligibility is verified at the time of patient SSO authentication; (8) patient benefits are determined by SSO authentication to a payor portal/dashboard; (9) providers can indicate whether they are accepting new patients at a regular interval (e.g., 60 days) or as required by law; (10) different stakeholders' information can be combined with insurance, medical history and attributed for patient referral (e.g., 3M® or Invisalign® can refer patients to providers with validated insurance information without having complicated BAA and HIPPA agreements with all the payors)—this mechanism does not currently exist; (11) HR133 compliance—consolidated information from unrelated activity used to update information to insurance companies to help facilitate compliance; (12) provider information can be searched across multiple lists (e.g., find a doctor tools, find a member lists, provider finder tool, etc.) and compared for inconsistencies; (13) information is securely and timely delivered to necessary stakeholders in timely concurrent processes automated by computer systems; (14) overcomes fragmented process that have happened in series, but with the call to action button (i.e., “book now”) these processes can happen timely and more accurately in one click; (15) moving data with one click simultaneously; and (16) data consolidation from not obvious bedfellows to help make the process better for everyone.

Other aspects of the present invention will become apparent with further reference to the following drawings and detailed description.

### BRIEF DESCRIPTION OF THE SEVERAL VIEWS OF THE DRAWINGS

For an improved understanding of the present invention, and the advantages thereof, reference is made to the following descriptions taken in conjunction with the accompanying figures:

FIG. 1 is a flowchart showing the overall organization and connection of patient, provider, payor, allied partner, system provider, and medical records company, to accomplish the secure management of transactions.

FIG. 2 shows a block diagram of a sequence for verifying provider practice information.

FIG. 3 shows a block diagram of a process of payor/organization updated list of providers through a CSV document; and a process of payor/organization onboarding through API.

FIG. 4 shows a block diagram of an asynchronous process of validating provider information through multiple sources.

FIG. 5 shows a block diagram of a sequence for securely sharing patient medical history.

FIG. 6 shows a block diagram of a sequence for securely sharing patient payment information.

FIG. 7 depicts a request for appointment.

FIG. 8 depicts a confirmation of requested appointment.

FIG. 9 depicts a booking of appointment.

FIG. 10 depicts a booking of appointment through third party integration.

FIG. 11 depicts a confirmation of booked appointment

FIG. 12 depicts a provider token definition object.

FIG. 13 depicts a request to verify provider info.

FIG. 14 depicts a request to update provider info.

FIG. 15 depicts a provider token definition object.

FIG. 16 depicts a request for patient payment data.

FIG. 17 depicts a request for patient medical history.

FIG. 18 depicts a session token of an appointment request.

FIG. 19 depicts a session token of an appointment confirmation.

FIG. 20 depicts a session token of a patient medical history request.

FIG. 21 depicts a session token of a patient payment data request.

FIG. 22 depicts a session token of a provider verification.

FIG. 23 depicts a session token of an update to provider information.

FIG. 24 depicts a patient identifier object.

FIG. 25 depicts a provider identifier object.

FIG. 26 depicts a payor identifier object.

FIG. 27 depicts a partner identifier object.

### DETAILED DESCRIPTION OF THE INVENTION

The present invention simplifies the problems that arise with the secure exchange of information between healthcare stakeholders (e.g., patients, providers, payors, and allied partners). The present invention facilitates the stakeholders timely receiving the info they need (and restricting info they do not need) with consistency, clarity, accuracy, and reliability.

FIG. 1 is a flowchart showing the overall relationship and exchange of information between a patient 30, a provider 32 (e.g., physician, dentist, etc.), a payor 34 (e.g., insurance carrier), an allied partner 36 (e.g., medical device manufacturer), and a system of secure data aggregation and distribution (“SOSDAD”) 38. An arrow points from patient 30 towards call to action 40. In this embodiment, by selecting call to action 40, the patient 30 starts the sequence of requesting and booking an appointment with a provider 32. After clicking the call to action 40, the patient is shown an up-to-date list of providers 32. This data flows through the SOSDAD 38 to a provider 32.

In one system embodying the invention, when the patient 30 clicks the call-to-action button 40, it triggers a cascade of actions. First, a patient makes an appointment via a scheduling application. Attribution information (i.e., sources of information such as referral sources, 3M®, Crest®, Blue Cross®) passes between payor and the provider. Simultaneously, patient authorized demographic information (e.g., address, phone, email, emergency contact, universal Medical HX form, permission(s)) conveys.

Next, the provider confirms the appointment and provides provider information (e.g., appointment information, hours of operation, address of provider, pricing information, etc.). Next, the payor confirms that the provider is accepting new patients. Patient information then passes from the payor to the provider.

In one system embodying the invention, the payor provides the patient with the network/provider panel (e.g., a list of providers in network). Second, the provider verifies federally mandated directory data. Third, patient information in the form of consolidated data for federally mandated HR133 is communicated to the payor. Fourth, affiliated partners introduce patients into the system. Fifth, the system consolidates attribution of referrals and use.

The requested appointment, the information for the appointment such as the provider's schedule and any other information that may be required in order to book an appointment with the provider (e.g., location of the provider, hours of operation, whether the provider is accepting new patients, whether the provider wants to receive digital insurance information from the SOSDAD, whether the provider wants to receive digital patient information from the SOSDAD (e.g., medical history)) is gathered ahead of time from the provider during onboarding and/or third party integrations (e.g., CSV and API from third-parties such as payors and allied partners). Because the SOSDAD 38 knows the provider's schedule and is able to interact with it, the SOSDAD 38 can provide patient 30 with a real time list of times that provider 32 is available.

The SOSDAD 38 obtains the provider's availability through an integration between the practice management software and our system via API. For example, the provider 32 can make her schedule accessible to the SOSDAD 38 through a combination of API of the provider's practice management software and the SOSDAD's API. The SOSDAD can then read the provider's 32 calendar and share it with prospective patients. In addition to reading, the SOSDAD can also write the patient-provider appointment into the provider's calendar, assuming the provider has provided the SOSDAD with access to the provider's calendar.

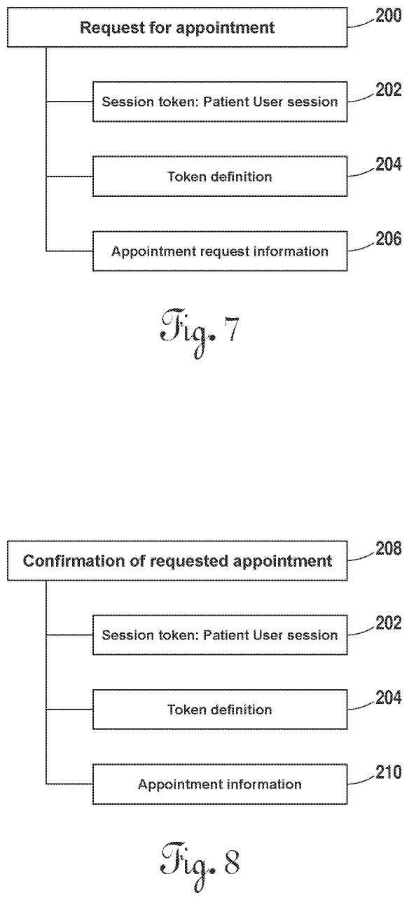

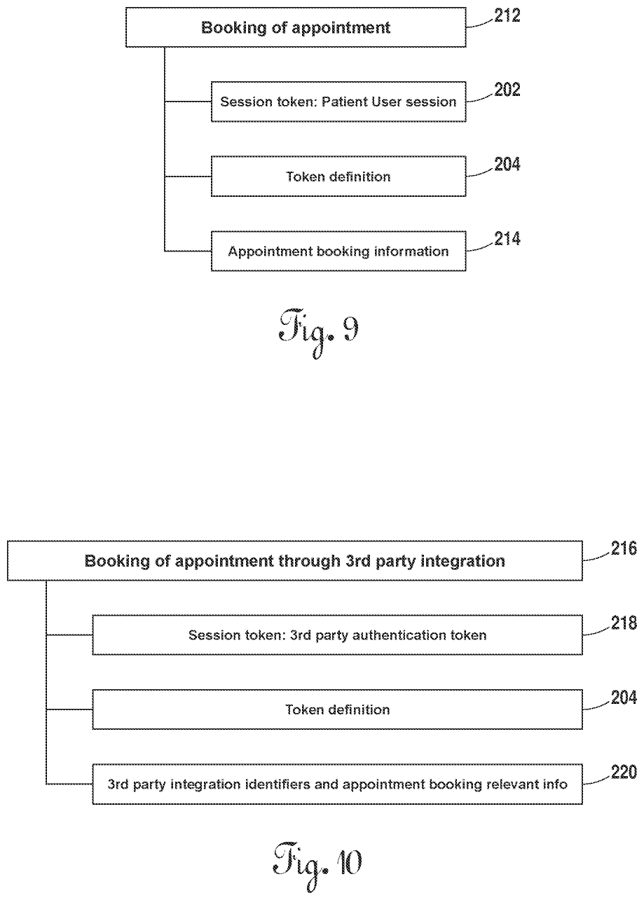

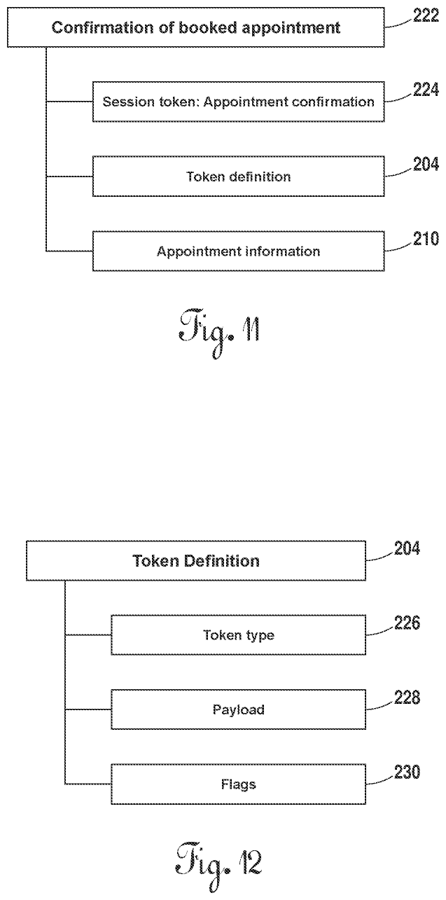

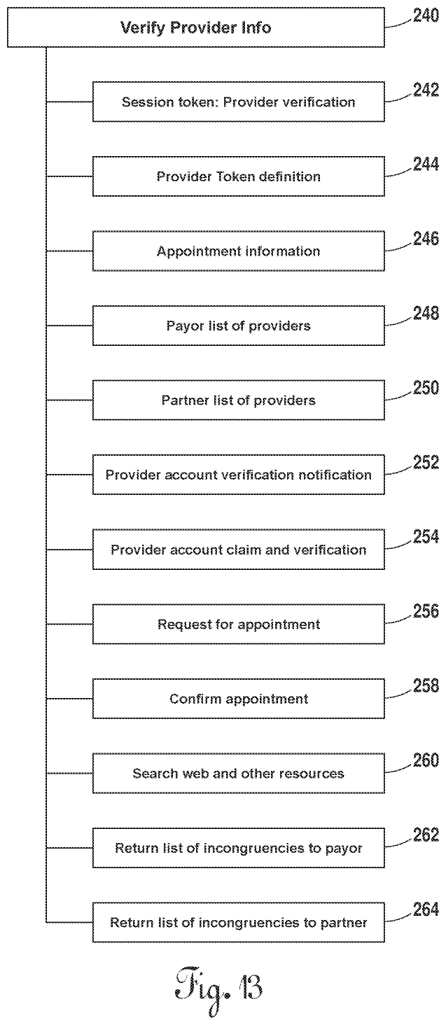

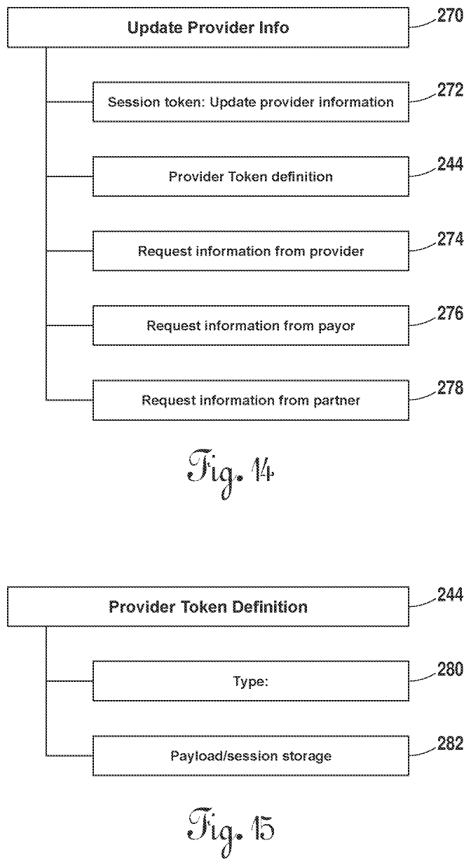

After the patient 30 selects an appointment time with the provider 32, the SOSDAD 38 can ask the patient for additional information requested by the provider 32 (e.g., birthday, names of children, insurance information and coverage, whether the patient consents to digital transmission of insurance information, whether the patient consents to digital transmission of medical history). In this embodiment, and as discussed below (and shown in FIGS. 5 and 6) the SOSDAD 38 provides a mechanism allowing patients to transfer this information to the provider securely through the SOSDAD 38.

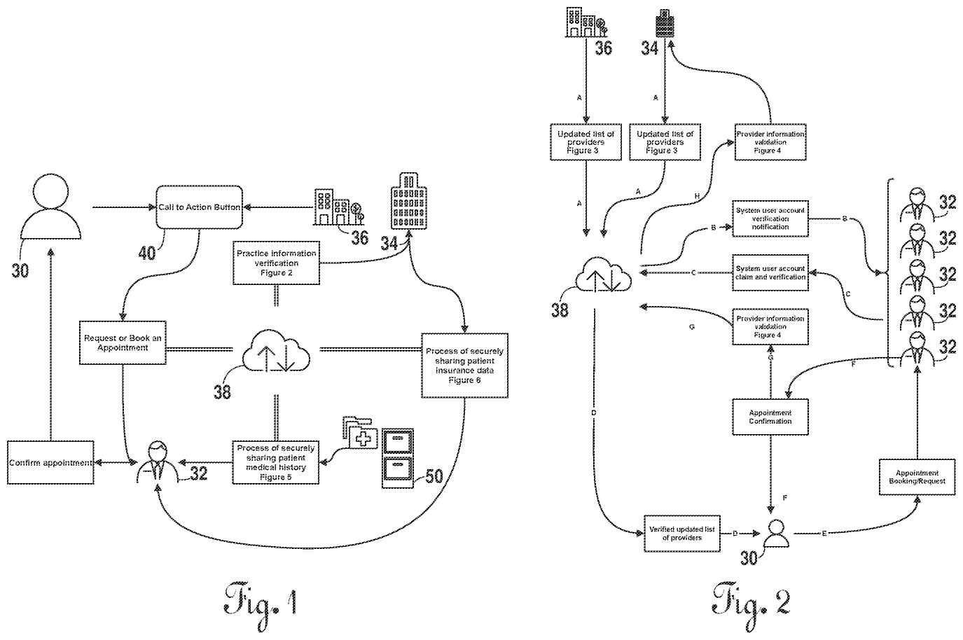

FIG. 2 illustrates an embodiment wherein the patient 30 is asked to provide additional information requested by the provider 32. For example, if the provider has indicated to the SOSDAD 38 that the provider wishes to receive medical history and/or insurance information digitally, the patient is asked whether he consents to share medical history and/or insurance information digitally. The patient 32 can do this asynchronously. More specifically, if the patient is booking an appointment outside of the operating hours of the provider's practice, the patient can book an appointment and provide information without having to do so during the provider's practice operating hours. Stated differently, the patient need not speak to a receptionist or otherwise book an appointment during operating hours.

At the time of booking the appointment, the SOSDAD 38 can provide payor 34 information belonging to the patient 30 with the provider 32. In this embodiment, the only interaction between the patient and the SOSDAD 38 at the time of booking, is that the patient 30 consents to sharing his medical history and/or insurance information (i.e., payor information) digitally. There may be more information that a provider may ask the patient for (e.g., medical history) where the SOSDAD 38 could involve another third-party such as a medical records company 50 or facilitator 52. If the patient gives consent, the medical records company 50 or facilitator 52 can securely share medical history information with the provider 32.

The mechanism is similar to how the SOSDAD 38 shares information between the payor 34 and the provider 32—discussed further below—where the exchange of information would be between the medical records company 50 or facilitator 52 and the provider 32.

In this embodiment, the provider 32 has given permission to receive patient information through an API or a process delivered to them (i.e., a dashboard sharing the information). The patient 30 has given permission to pass that information through the SOSDAD 38 to the provider 32. In the case of the provider, the SOSDAD 38 provides the provider with a prepared form for the provider to give her consent. Likewise, the SOSDAD 38 provides the patient with a prepared form (e.g., HIPAA agreement) for the patient to give his consent.

These several actions (e.g., booking appointment (explicit), establishing appropriate BA's (business agreements), requesting medical history information, delivering medical history information, requesting payor information, delivering payor information, delivering the appropriate data to the appropriate stakeholder) happen simultaneously once the patient requests an appointment. In particular, and described in greater detail below, some of these actions are implicit and some of these actions are explicit. Implicit things happen because some prior authorization, whereas explicit actions require further approval or action by the stakeholder.

When the provider confirms the appointment with the patient, another process initiates, which verifies the provider's 32 information. The SOSDAD 38 verifies the provider's 32 information such as the provider's address, whether or not she is accepting new patients, whether the provider agrees to receive medical and/or payor information, digitally. The SOSDAD 38 supplies this information to one or more payors 34, for their legal compliances or demonstrates intent to comply by presenting the most accurate information about the provider, namely, to comply with HR 133 with respect to payor transparency.

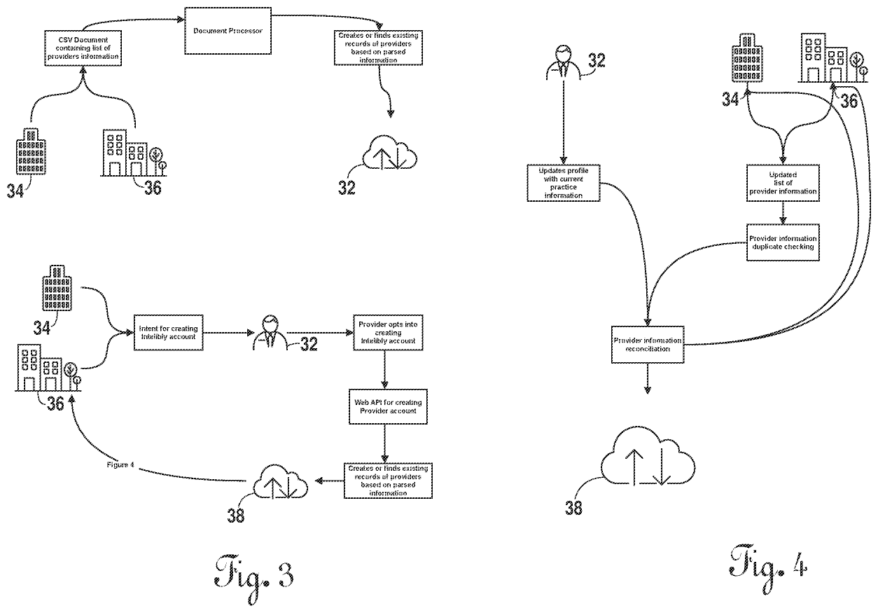

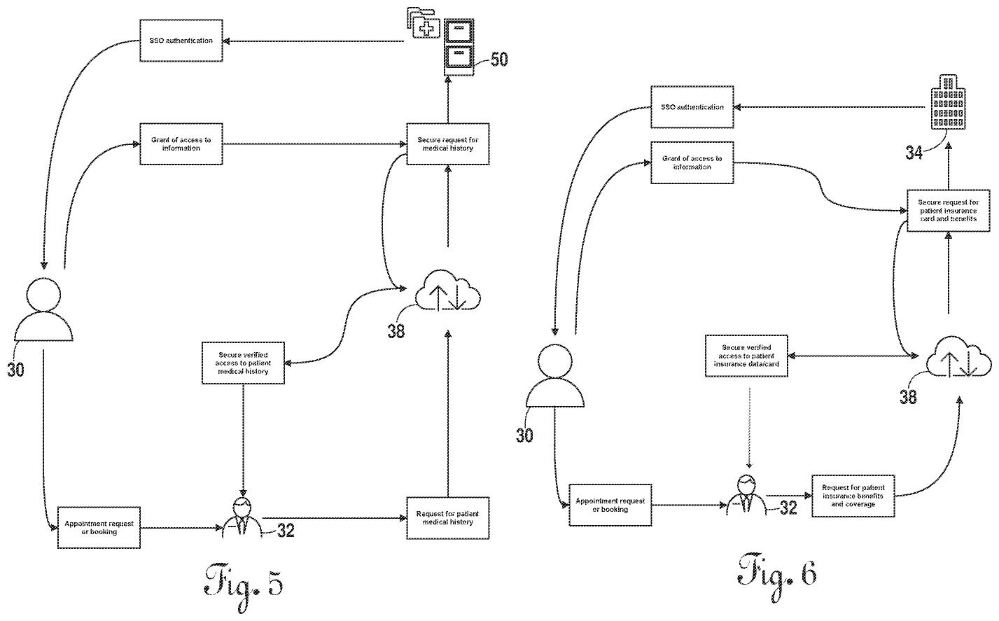

FIG. 2 shows the process of continuous provider information verification. Some of the steps can occur synchronously and other steps must occur asynchronously. Steps A-C occur synchronously and steps D-H occur asynchronously. The process of continuous provider information verification starts with payor 34 and allied partner 36 providing the updated list of providers to SOSDAD 38, which is illustrated in more detail in FIG. 3. In response to payor 34 and allied partner 36 providing the updated list of providers to SOSDAD 38, SOSDAD 38 will create a system user account verification (Step C) as a notice to the provider(s) 32. This could be a single provider, or this could be a plurality of providers provided to the SOSDAD 38 by payor 34 and allied partner 36. To accomplish this step, the SOSDAD 38 provides payor 34 and/or allied partner 36 with a mechanism for uploading a list of providers. The present embodiment shown in FIG. 4 illustrates a group of five providers 32. Each provider will individually verify his/her system account. The system account is unique to each provider and allows the provider to securely access and share data (e.g., appointment information, payors that the provider works with, patient medical history that the provider has not yet accepted from the SOSDAD but needs to determine whether it will download to the provider's internal system). Verification may occur in the present embodiment when the SOSDAD adds the provider based on lists from a payor or allied partner. The provider is prompted to claim her account and onboard with the SOSDAD. The verification communicates to the SOSDAD 38 that the provider's 32 information is correct and up to date. In particular, the provider's 32 information is verified with the SOSDAD 38 on an automated basis. Subsequently, the provider's information is verified with the SOSDAD 38 by way of interactions between the stakeholders (e.g., patients booking appointments, providers accepting appointments, providers logging in and updating personal system accounts, payors and allied partners providing updated lists of providers) and the SOSDAD 38.

The provider 32 verifying his/her system account allows the SOSAD 38 to present a verified updated list of providers to the patient 30. With the verified updated list of providers, the patient can book an appointment with provider 32. That will act as a mechanism of verification by the act of confirming that appointment with the provider. Stated differently, the booking of the appointment and the subsequent confirmation of the appointment by the provider acts as a mechanism of verification. With this confirmation, the provider's information is verified and flows back through step G to the SOSDAD 38 (explained in FIG. 4 and discussed below). A feedback loop results between the payors, the allied partners, the patient, and the provider that all feed accurate provider information into the SOSDAD 38.

For example, during the Step C of system user account claim and verification: when the provider 32 verifies her account she also agrees to the terms and putting in her preferences (e.g., does she want to receive medical histories, does she not want to receive medical histories, does she work with this insurance company, does she not work with this insurance company, etc.), the provider verifies her information (e.g., her name, her address, whether information about her practice is correct, what information she is willing and/or authorizing to receive, whether she agrees to SOSDAD 38 terms per HIPPA, privacy, PIP privacy patient information). This information is stored by SOSDAD 38 but not exchanged.

The Step C of system user account claim and verification also establishes the necessary legal documents and agreements that SOSDAD 38 needs to have as a business entity operating with this provider and sharing HIPPA information. Stated differently, the provider 32 and SOSDAD 38 execute the necessary business agreement (e.g., HIPPA consents, etc.). All of that happens in that moment. It could occur when a provider onboards with SOSDAD 38 or a provider who has an existing account with SOSDAD 38 but is being added to a new payor 34 network. In the latter instance, perhaps, there are additional agreements being set up at that time.

FIG. 42 is the channel that is being communicated here. It is drawn as an arrow coming out of 38 into 34 because there are some processes that are implicit and some processes that are not illustrated herein and just abbreviated as FIG. 2.

The No Surprises Act (H.R. 133) seeks to protect consumers from surprise medical bills arising out of certain out-of-network emergency care. The invention described herein can be used to comply with H.R. 133 by way of regularly updating and verifying party information. For example, H.R. 133 requires verification of provider information every three months. The frequency is inherently more often than that but could be adjusted if the law changes or the payor requests. Step H of delivering and verifying the provider's 32 information back to the payors 34 demonstrates the pattern of compliance for HR133.

Some of the steps in FIG. 2, which shows the process of continuous provider information verification, are implicit.

FIG. 3 describes the mechanism of delivering SOSDAD 38 the updated list of providers from the allied partners 36 and the payers 34. In this embodiment, the delivery occurs either through a comma-separated values (CSV) document or onboarding through API.

With respect to the CSV, both payor 34 and allied partner 36 have a comma-separated value document(s) containing a list of providers that each provider shares with the SOSDAD 38 by way of the SOSDAD's 38 document processor. The document processor is a proprietary process of data import and information verification. The CSV can identify a plurality of providers and contain various information such as the provider's name, her office address, her phone number, her email address, etc.

When the payor 34 or partner 36 delivers the CSV document to SOSDAD 38, the payor 34 or partner 36 implicitly or explicitly consents to SOSDAD 38: importing this data into its system, contacting the providers, and creating businesses agreements with each provider so they can start using the SOSDAD's 38 system.

The document processor (i.e., importer) is a unique process for running a CSV file and creating provider accounts with the SOSDAD 38. The document processor performs several steps: verifying the information, checking for duplicates, validating to make sure that the email addresses are deliverable, validating that the physical address is an actual physical location in the world. The SOSDAD's 38 proprietary software processes supplied provider information and creates a provider account that is unique to the provider. The output of the document processor is the creation or finding of several existing records for providers based on the information that was given in that CSV document.

After the CSV document is processed, the present invention may comprise an onboarding step. The onboarding step is less systematic than the step of processing the CSV document. The onboarding step could be characterized as a sequence of steps to encourage the provider(s) to complete the onboarding process with SOSDAD 38.

Alternatively to the step of processing the CSV document, the API assembles all of those steps and performs the steps more automatically. In the case of an API onboarding, both the payer or the organization if they have a dashboard for their provider, they can create a page that can initiate the intent for creating an account with SOSDAD 38. For example, when a provider goes through onboarding and receives a message along the lines of “sign up with SOSDAD 38” and clicks on that page, rather than asking the provider for all of the necessary information about her office locations, her payors, etc., SOSDAD 38 can request this information to be electronically transferred from the payor or the allied partner at that time. The only thing that the provider would have to do is grant SOSDAD 38 access to the information.

In essence, connecting to the API allows the provider to create an account with SOSDAD 38 without having to do anything other than saying “yes, create my account” (or the like). This occurs at the time the provider presses the button. There are no backward requests. As soon as the provider grants access, the web API system on the payer or partner side is going to make a web request to SOSDAD 38. The request contains a secured request containing all necessary information (e.g., the provider's practice name, the location address, the phone numbers, the email addresses, etc.). All of the necessary information is transferred from the payer or partner to SOSDAD 38 at that time. Once the provider consents/clicks the button/opts in, all that information is securely sent to SOSDAD 38. At that point, SOSDAD 38 can then create or find existing records of providers based on the parsed information. The SOSDAD 38 can search the Internet and/or databases, etc. for additional information. With the additional information, the SODSAD 38 can find incongruencies.

FIG. 4 illustrates communications sent from the SOSDAD 38 to the payer or partner for H.R. 133 compliance. H.R. 133 compliance occurs, at least in part, because SOSDAD 38 receives provider data from multiple sources. If there are incongruencies, SOSDAD 38 can update the different stakeholders and let them know that a stakeholder has a different address. Stated differently, and by way of example, SOSDAD 38 automatically notifies the stakeholder to say “your address is different from what other people have for this provider.” This process is the asynchronous process of validating provider information through multiple sources.

This process occurs asynchronously, because the SOSDAD 38 continuously receives new information from the payers and the allied partners in a form containing updated CSV documents (i.e., the list containing provider information) or an API request. The providers information is checked for duplicates and then it goes through a provider information reconciliation process.

A provider can update her provider information with SOSDAD 38 using the SOSDAD 38's provider interface/dashboard. Presumably, the provider will provide SOSDAD 38 with the most up-to-date and most accurate information about her practice. That is being fed into the provider information reconciliation. Through provider information reconciliation, SOSDAD 38 can make several decisions about whether the information SOSDAD 38 has from the provider is accurate. The SOSDAD's 38 records indicate when the provider information was updated. SOSDAD 38 knows whether the provider has recently logged in and/or recently confirmed or updated her information. SOSDAD 38 can require an explicit acknowledgement from the provider to tell SOSDAD 38 whether provider has reviewed the information, checked for accuracy, and confirm that the information is accurate.

SOSDAD 38 can then generate a report based on the information received from or regarding the payor and/or allied partner to see if there are any incongruencies. For example, SOSDAD 38 can highlight incongruent information and provide a report back to the payor and/or allied partner in a feedback loop to let them know information that they have on record is inaccurate based on the information SOSDAD 38 received directly from the provider, payor and/or allied partner and when it was verified and organized. SOSDAD 38 can deliver this report to the payor and/or allied partner on a regular interval (e.g., quarterly basis, weekly basis, daily basis, etc.).

FIG. 5 illustrates the process of securely sharing patient medical information When the patient schedules an appointment, patient 30 requests an appointment with the provider 32, and the provider 32 has already consented to SOSDAD 38 that provider 32 is willing to receive the patient's medical history electronically (or any prospective patient's medical history, for that matter). On provider's behalf, SOSDAD 38 securely creates a request for medical history information as illustrated by the arrow going from SOSDAD 38 to medical records company 50. Specifically, SOSDAD 38 requests the patient's 30 medical history information from the medical records company 50 or facilitator 52.

This secure request is used to facilitate the secure transfer of this information with the patient's consent. As the patient makes the request for an appointment, the request for medical history is sent to the medical record facility, medical record system to request this information for this patient. In turn, the medical record system will create a single sign-on authentication screen for the patient, allowing them to log in securely into their system. When logged in, the patient is prompted to grant access to the information about their medical history. That could include information such as the patient's most recent procedures, medication use. This information is important and relevant to the provider, and it is securely kept within the SOSDAD 38. This information is attached to the initial request so that SOSDAD 38 can guarantee that no one will have access to the information except for the entity that requested it. In this case, the provider. SOSDAD 38 holds onto this information securely and we can only disclose this information once the provider has explicitly asked for it. The provider implicitly indicated to SOSDAD 38 that she wants the patient's medical history information. Because this process can happen asynchronously, the patient may request an appointment when the office is closed.

A similar sequence occurs with respect to the exchange of patient insurance information. The patient is allowed to explicitly grant permission for SOSDAD 38 to retrieve and forward insurance information at the time that the patient books the appointment. Once consent is given, SOSDAD 38 retrieves the insurance information, and the information resides within SOSDAD's 38 system temporarily until the provider is ready to view the information. Once the provider requests access to the patient's insurance information, it is securely erased from SOSDAD's 38 system and is completely in the possession and control of the provider 32. It minimizes the amount of time he spends at the office when he arrives for his appointment. The secure transfer of information and the flow are exactly the same.

The SOSDAD 38 may maintain additional information while securely holding the medical history or insurance information. SOSDAD 38 is aware of the initial request and can verify with high accuracy who has access to this information and when. The SOSDAD 38 carries out secure and accurate transfers without allowing any third party to be able to see the patient information unintentionally. Unintentional disclosure is more likely to occur with human involvement than the present invention.

Each instance that the provider interacts with SOSDAD 38, there is another chance to verify that the provider's information is correct and to comply with H.R. 133.

FIG. 6 illustrates the process of securely sharing the patient's insurance information. The patient expressly requests or books an appointment with the provider. The provider has previously granted SOSDAD 38 permission or otherwise notified SOSDAD 38 that she is interested in receiving this information digitally. On provider's behalf, SOSDAD 38 will make a request to the payer 34 to request insurance card or benefits information. At that time the payer will present a single sign on authentication screen to the patient so that they can log on to their system and then grant access to this information. The insurance information might include the patient's insurance number, group number, benefits, coverage, and other information that might be necessary for the provider to keep on record. This information is kept securely in the SOSDAD's 38 system while SOSDAD 38 waits for the provider to request access. In some embodiments, there may be a time limit on how long the SOSDAD 38 is able to keep this information, as an additional security measure. Once the provider requests access to this information it is in her possession and control, the information is securely erased from the SOSDAD's 38 system.

Note that the SOSDAD 38 eliminates direct communication between the provider directly and the payor at the time of booking. Doing so vastly improves the security of information. There are no HIPPA breaches of HIPPA, cybersecurity risks are minimized, and permissions are in place.

The SOSDAD 38, by way of the invention, facilitates the secure transfer of patient information. The SOSDAD 38 guarantees the accountability of the information being accessed by the party who it is intended for. The invention comprises extra steps when there is a request for information from the provider side: SOSDAD 38 explicitly creates a single time use secure request for this patient information. When the grant is given explicitly by the patient, it is attached to this request so extra validation happens between the request and the grant making sure that this is not a stale request—not from the two or three times before they asked. But this is the most recent request and it is validated by this provider and this provider is still in business and the SOSDAD can make sure that she is who she says that she is—before the SOSDAD 38 allows the information to be accessed.

FIG. 7 depicts a request for appointment 200 in an alternative embodiment of the invention. The request for appointment 200 comprises a Session token: patient user session 202, a token definition 204, and an appointment request information 206. The request for appointment is an act by patient user of requesting the practice user (e.g., one or more employees of the provider) to reach out to the patient user with available appointment slots and subsequent booking of one of those slots. In this alternative embodiment, the booking of appointment will happen outside of SOSDAD 38. The Session token: patient user session 202 could be a session in alternative embodiments, but not in the primary embodiment described above. The appointment request information 206 comprises information about the patient, location of the appointment, date/time details of the appointment.

FIG. 8 depicts a confirmation of requested appointment 208. The confirmation of requested appointment 208 comprises a session token 202, a token definition 204, and appointment information 210. Appointment Information 210 is not part of the session in several embodiments because the confirmation will be accessible via email, text message, or 3rd third-party dashboard not relating to SOSDAD.

FIG. 9 depicts a booking of appointment 212. The booking appointment 212 comprises a session token: patient user session 202, a token definition 204, and an appointment booking information 214. The session token: patient user session 202 could be a session in certain embodiments of the invention but not all embodiments. Appointment booking information 214 comprises information about the patient, location of the appointment, and date/time details of the appointment.

FIG. 10 depicts a booking of appointments through third-party integration 216. Booking of appointment through third-party integration 216 comprises a session token: third-party authentication token 218, a token definition 2014, and third-party integration identifiers and appointment booking relevant information 220. Session token: third party authentication token 218 is a session generated for SOSDAD by a third-party integration provider in order to securely transmit appointment booking information. The token definition is a JSON Web token (JWT) which is generated for the session between SOSDAD and third-party integration using pre-shared secure credentials, or Security Assertion Markup Language (SAML) token or Simple Web token (SWT). Third-party integration identifiers and appointment booking relevant information 220 is information transmitted to 3rd party integration, which may include: 1) SOSDAD unique identifier number 2) Patient's personally identifiable information 3) Appointment relevant information such as insurance provider name, plan information, treatment information (data that 3rd party integration requires SOSDAD to ask the patient for).

FIG. 11 depicts a confirmation of booked appointment 222. Confirmation of booked appointment 222 comprises a session token: appointment confirmation 224, a token definition 204, and appointment information 210. Confirmation of booked appointment 222 is a response to the booking of appointment and is accessible via patient user session with the SOSDAD. The patient user can also be notified by SOSDAD via email/text message or via 3rd party integration (without involvement of SOSDAD). The session token: appointment confirmation 224 is a session in some embodiments but not all. The appointment information object 210 is not part of the session in several embodiments because the confirmation will be accessible via email, text message, or 3rd party dashboard not relating to SOSDAD.

FIG. 12 depicts a provider token definition object 204. The token definition object 204 comprises a token type 226, a payload 228, and one or more flags 230. It is contemplated that the token type 226 is JWT or other industry standard mechanisms. See, e.g., https://auth0.com/learn/json-web-tokens. The payload 228 is any of a session identifier, patient user identifier. Token flags can have arbitrary names and purpose based on context.

FIG. 13 depicts a request to verify provider info 240. A request to verify provider info 240 comprises Session token: Provider verification 242, Provider Token definition 244, Appointment information 246, Payor list of providers 248, Partner list of providers 250, Provider account verification notification 252, Provider account claim and verification 254, Request for appointment 256, Confirm appointment 258, Search web and other resources 260, Return list of incongruencies to payor 262, and Return list of incongruencies to partner 264.

A session exists when accessing account claim and verification. Requests for appointments, confirmations of appointments. Appointment information 246 is information about available appointment slots at a given provider location for the given provider. Payor list of providers 248 is a payor supplied list of provider information including location address, contact information, provider name, provider specialty, national provider identifier, other provider information, practice email address, provider email address, other payor supplied information such as provider participating plans etc. Partner list of providers 250 is a partner's supplied list of provider information similar to payor but without the payor/insurance specific information. It may also include partner specific information such as provider product participation etc. Provider account verification notification 252 is an email notice/phone call/direct mail letter notifying the provider about SOSDAD account creation or account update. Provider account claim and verification 254 is a webpage part of SOSDAD asking the provider to review and verify their information which was provided by Payor or Partner. Request for appointment 256 is a request for appointment can be either a Request for Appointment or Appointment Booking. It contains information collected from a patient during the patient's appointment booking or request for appointment. Confirm appointment 258 is an acknowledgement and a confirmation of request for appointment or an appointment booking. Search web and other resources 260 comprises resources for verifying provider information correctness can include 3rd party API's and or databases of provider information as well as SOSDAD information. Return list of incongruencies to payor 262 is provider information that SOSDAD has reviewed and verified to be incongruent with information provided by the payor. Return list of incongruencies to partner 264 is provider Information that SOSDAD has reviewed and verified to be incongruent with information provided by the partner.

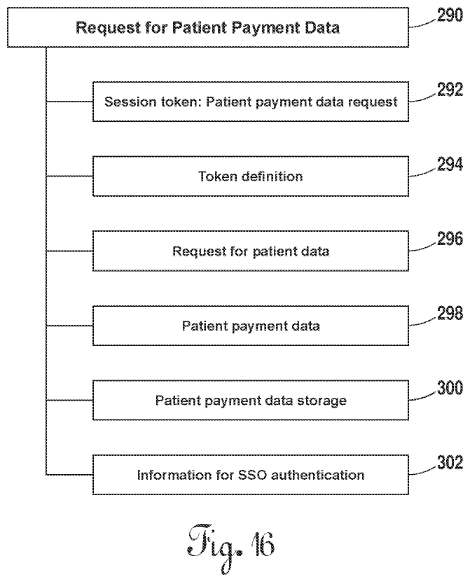

FIG. 14 depicts a request to update provider info 270. Request to update provider info 270 comprises a session token: update provider information 272, a provider token definition 244, a request information from provider 274, a request information from payor 276, and a request information from partner 278. A request information from provider 274 is information provided directly by the provider about the location address, contact information etc. A request information from payor 276 is information provided directly by the payor about the location address, contact information etc. of the provider. A request information from partner 278 is information provided by the partner about the provider's location address, contact information etc.

FIG. 15 depicts a provider token definition object. A provider token definition object 244 comprises a type 280 and payload/session storage 282. The type 280 is a JWT or a cookie. The payload/session storage 282 is a provider identifier.

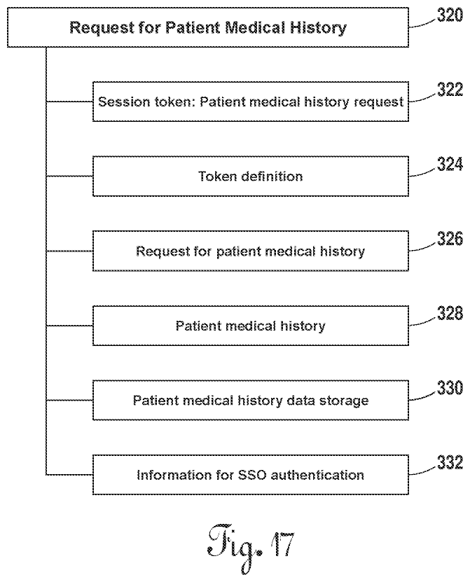

FIG. 16 depicts a request for patient payment data 290. A request for patient payment data 290 comprises a session token: patient payment data request 292, a token definition 294, a request for patient data 296, patient payment data 298, patient payment data storage 300, and information for SSO authentication 302. Regarding the session token: patient payment data request 292, there might be a combination of sessions involved, one session from the patient initiating the SSO authentication, one session from the provider accessing the requested information. A request for patient data 296 is a time constrained unique request for patients payment information which can either be fulfilled or rejected with patient's consent. Patient payment data 298 refers to the information transmitted from payor to provider containing information such as insurance card, available benefits, deductible information etc. Patient payment data storage 300 refers to the mechanism of securely storing the patient payment information in encrypted state until the provider accepts to receive this information. This information is housed temporarily and with special policies for provider. The storage mechanism knows about the initial request for patient data the subsequent fulfillment or rejection. Once this information has been accessed or copied by the provider SOSDAD records the time and date of this access and removes the patient payment data from the storage. Regarding information for SSO authentication 302, during SSO authentication between patient and payor the patient provides secure credentials to the payors portal.

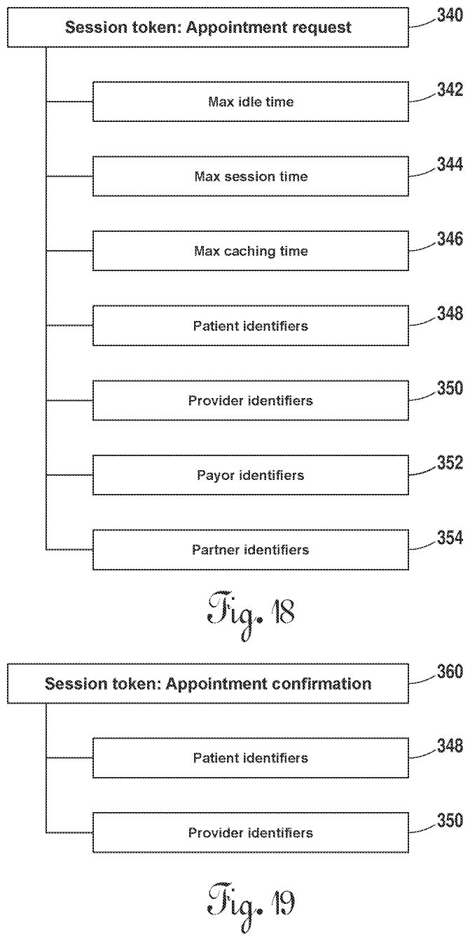

FIG. 17 depicts a request for patient medical history 320. A request for patient medical history 320 comprises a session token: patient medical history request 322, a token definition 324, a request for patient medical history 326, patient medical history 328, patient medical history data storage 330, and information for SSO authentication 332. Regarding the session token: patient medical history request 322, there might be a combination of sessions involved, one session from the patient initiating the SSO authentication, one session from the provider accessing the requested information. A request for patient medical history 326 is a time-constrained unique request for a patient's medical history, which can either be fulfilled or rejected with the patient's consent. Patient medical history 328 refers to the information transmitted from medical records company containing information concerning the patient's medical history. Patient payment data storage 330 refers to the mechanism of securely storing the patient medical history in encrypted state until the provider accepts to receive this information. This information is housed temporarily and with special policies for provider. The storage mechanism knows about the initial request for patient data the subsequent fulfillment or rejection. Once this information has been accessed or copied by the provider, the SOSDAD records the time and date of this access and removes the patient medical history from the storage. Regarding information for SSO authentication 302, during SSO authentication between patient and medical records company, the patient provides secure credentials.

Token definition 294, 324 is JWT or other industry standard mechanisms. See, e.g., https://auth0.com/learn/json-web-tokens.

FIG. 18 depicts a session token of an appointment request 340. A session token of an appointment request 340 comprises a max idle time for the appointment request 342, a max session time for the appointment request 344, a max caching time 346, patient identifiers 348, provider identifiers 350, payor identifiers 352, and partner identifiers 354. Industry standard times will apply in several embodiments of the invention with respect to max idle time for the appointment request 342, max session time for the appointment request 344, max caching time 346 (e.g., 10 minutes, 1 hour, 1 hour, respectively), although that is not mandatory. Patient identifiers 348, provider identifiers 350, payor identifiers 352, and partner identifiers 354, as discussed below, are universally unique identifiers (UUID) for patients, providers, payors, and partners, respectively, assigned by the SOSDAD. The patient identifier 348 may be information provided by a patient without an active session.

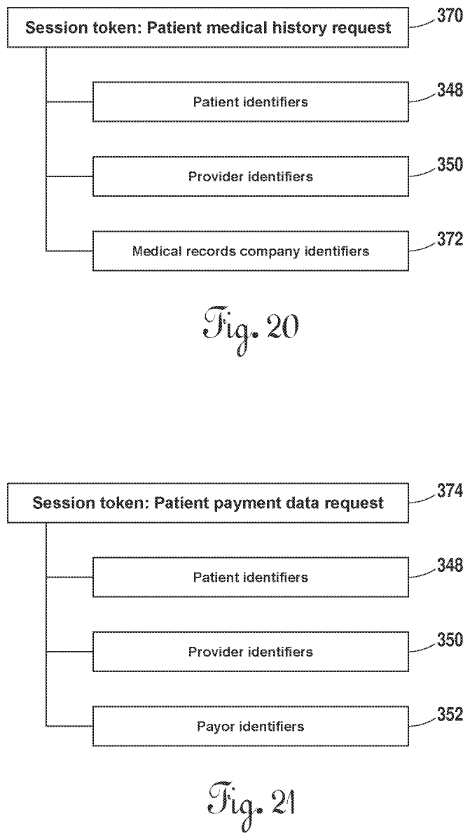

FIG. 19 depicts a session token of an appointment confirmation 360. A session token for appointment confirmation 360 comprises patient identifiers 348 and provider identifiers 350 if a session is initiated.

FIG. 20 depicts a session token of a patient medical history request 370. A session token for patient medical history request 360 comprises patient identifiers 348, provider identifiers 350, and medical records company identifiers 372. Medical records company identifiers 372 are universally unique identifiers (UUID) for a medical records company assigned by the SOSDAD.

FIG. 21 depicts a session token of a patient payment data request 374. A session token for patient payment data request 374 comprises patient identifiers 348, provider identifiers 350, and payor identifiers 352. Payor identifiers 352 are universally unique identifiers (UUID) for a payor assigned by the SOSDAD.

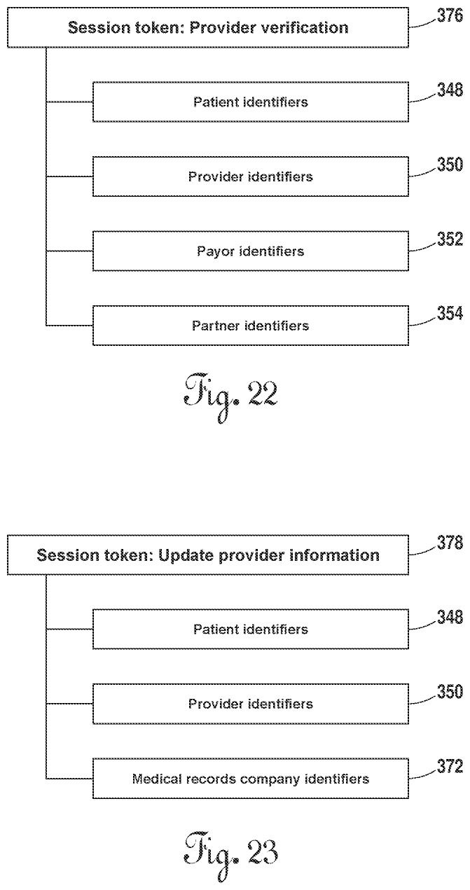

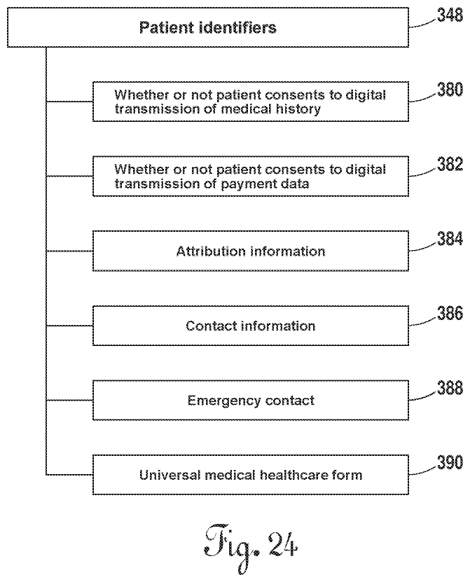

FIG. 22 depicts a session token of a provider verification 376. A session token for provider verification 376 comprises patient identifiers 348, provider identifiers 350, payor identifiers 352, and partner identifiers 354. In some embodiments, the session token of a provider verification 376 will not comprise both payor identifiers 352 and partner identifiers 354.

FIG. 23 depicts a session token of an update to provider information 378. A session token for provider verification 378 comprises patient identifiers 348, provider identifiers 350, and medical records company identifiers 372.

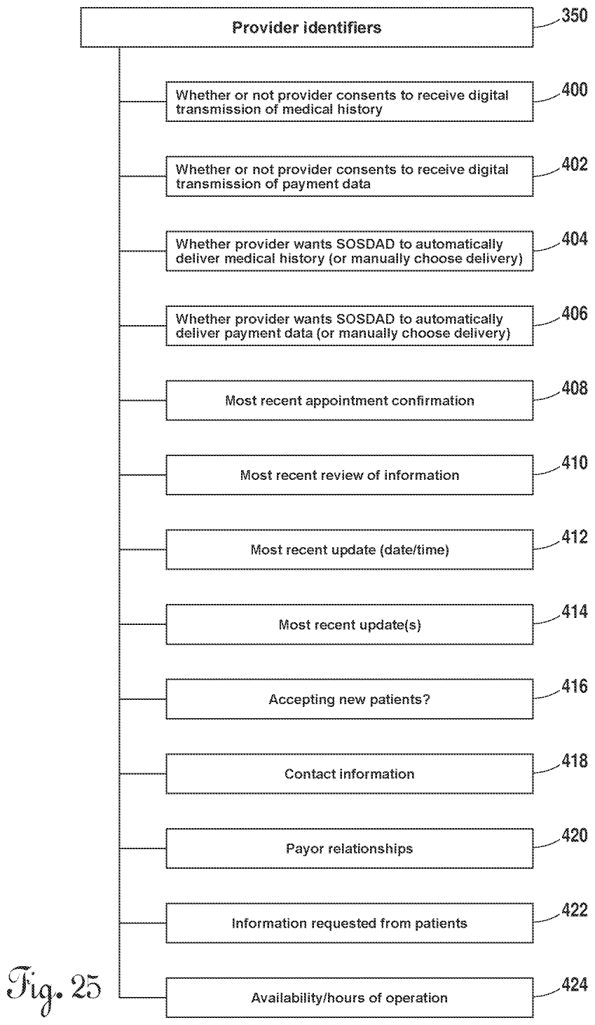

FIG. 24 depicts a patient identifier object 348. A patient identifier object 348 comprises whether or not patient consents to digital transmission of medical history 380, whether or not patient consents to digital transmission of payment data 382, attribution information 384 (e.g., who referred the patient), contact information 386 (e.g., patient phone number, patient email address, etc.), patient emergency contact 388 (e.g., Sister Michelle and phone number), universal medical healthcare form 390.

FIG. 25 depicts a provider identifier object 350. A provider identifier object 350 comprises whether or not provider consents to receive digital transmission of medical history 400, whether or not provider consents to receive digital transmission of payment data 402, whether provider wants SOSDAD to automatically deliver medical history (or manually choose delivery) 404, whether provider wants SOSDAD to automatically deliver payment data (or manually choose delivery) 406, most recent appointment confirmation 408 (e.g., provider last confirmed an appointment 9 months ago; provider last confirmed a patient at 14:04 this afternoon), most recent review of provider information 410, most recent update to provider information (date/time) 412, most recent update(s) 414, whether provider is accepting new patients 416, contact information 418, payor relationships 420, information requested from patients 422, availability/hours of operation 424.

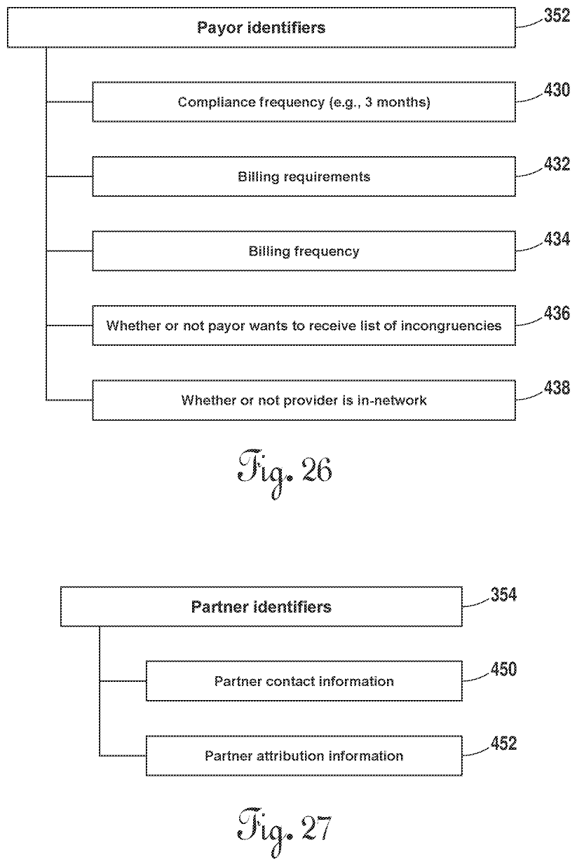

FIG. 26 depicts a payor identifier object 352. A payor identifier object 352 comprises compliance frequency 430 (e.g., 3 months), billing requirements 432 (e.g., formatting), billing frequency 434, whether or not payor wants to receive list of incongruencies 436, and whether or not provider is in-network 438.

FIG. 27 depicts a partner identifier object. A partner identifier object 354 comprises partner contact information 450, and partner attribution information 452.

The present invention is described above in terms of a preferred illustrative embodiment in which a specifically described refining plant and method are described. Those skilled in the art will recognize that alternative constructions of such an apparatus, system, and method can be used in carrying out the present invention. Other aspects, features, and advantages of the present invention may be obtained from a study of this disclosure and the drawings, along with the appended claims.

We claim:

1. A computer-implemented system for storing computer program instructions, which, when executed by a processor, cause the processor to perform a method of sharing and restricting data between healthcare stakeholders, the computer-implemented system comprising:
one or more servers in secure communication with a patient by way of a patient's computer, a provider's computer, a payor's computer, or any combination thereof, the one or more servers having:
a first instruction embodied in a non-transitory computer readable medium to receive a request for scheduling an appointment from a patient, the request containing an appointment request session token, and a received appointment request value, wherein the appointment request session token includes a policy identifier logically related to the patient, a policy identifier logically related to the provider;
a second instruction embodied in a non-transitory computer readable medium to receive a confirmation of an appointment from a provider, the request containing an appointment confirmation session token, and a confirmed appointment value, wherein the appointment confirmation session token includes a policy identifier logically related to the patient, a policy identifier logically related to the provider;
a third instruction embodied in a non-transitory computer readable medium to verify provider information;
a fourth instruction embodied in a non-transitory computer readable medium to update the provider information;
a fifth instruction embodied in a non-transitory computer readable medium to receive a request for providing patient medical history from a provider, the request containing a medical history session token, and a received medical history value, wherein the medical history session token includes a policy identifier logically related to the patient, a policy identifier logically related to the provider, a policy identifier logically related to the medical records company;
a sixth instruction embodied in a non-transitory computer readable medium to receive a request for providing patient payment information from a provider, the request containing a payment information session token, and a received payment information value, wherein the payment information session token includes a policy identifier logically related to the patient, a policy identifier logically related to the provider, a policy identifier logically related to the payor.

2. The computer-implemented method of claim 1 wherein the request contained in the third instruction contains a provider verification session token, and a received provider verification value, wherein the provider verification session token includes a policy identifier logically related to the patient, a policy identifier logically related to the provider, a policy identifier logically related to the payor.

3. The computer-implemented method of claim 2 wherein the provider verification session token includes a policy identifier logically related to the partner.

4. The computer-implemented method of claim 1 wherein the request contained in the fourth instruction contains a provider update session token, and a received provider claim value, wherein the provider claim session token includes a policy identifier logically related to the patient, a policy identifier logically related to the provider, a policy identifier logically related to the payor.

5. The computer-implemented method of claim 4 wherein the provider update session token includes a policy identifier logically related to the partner.
